(19) United States
(12) Patent Application Publication
(10) Pub. No.: US 2013/0059918 A1
(43) Pub. Date: Mar. 7, 2013

(54) Throat irritation relief beverage

(71) Applicant: Terry Skirzyk, Red Deer (CA)

(72) Inventor: Terry Skirzyk, Red Deer (CA)

(21) Appl. No.: 13/137,679

(22) Filed: Sep. 2, 2011

(51) Int. Cl. A61K 31/045

(52) U.S. Cl. 514/729

(57) ABSTRACT

A composition beverage and method for relieving a symptom of a sore throat of a user. The composition comprises an aqueous base and an effective amount of a throat soreness relieving agent. The relieving agent may comprise between 0.001% w/v and 0.02% w/v of menthol. The beverage comprises a water base and including between 0.001% w/v and 0.02% w/v of a throat soreness relieving agent. The method comprises the administration of a safe and effective amount of a composition comprising an aqueous base and an effective amount of a throat soreness relieving agent.

### BACKGROUND OF THE INVENTION

1. Field of Invention

The present invention relates to beverages in general and in particular to a beverage for use in suppressing or relieving symptoms associated with cough and cold.

2. Description of Related Art

Throat irritation and cough is a common symptom for many people. Cough may a side effect of being infected by many types of viruses or bacteria. Alternatively, a sore throat may have other causes such as trauma. The level of pain experience by a person having a sore throat can range from mild to, in some cases be extremely painful.

Fortunately, for many type of sore throats, no medical treatment is necessary to resolve the underlying condition. Such is the case in many types of viral or bacterial infections which often resolve on their own or in the case of minor superficial irritations to the throat area will heal on their own. In such situations, the person so infected must commonly wait for the infection to be controlled by the person's own immune system, or “run its course”. Disadvantageously in such cases, however, the symptoms associated with the infection must also endured by the person during the infection. Therefore, common treatment for such individuals primarily focuses on relieving the symptoms associated with the infection, including the sore throat.

Current treatments for sore throats have not been completely satisfactory. Previously used treatments have been to use a cough syrup administered in a relatively small dose (typically between 1 and 2 teaspoons) which includes a pain relieving medicine or cough suppressant. Such syrups are taken in a single dosage which is then supposed to be effective for a period of time. Disadvantageously, such a single dosage may not be effective at coating the entire throat and therefore may have limited effect in providing effective relieve to the affected area of the throat. Additionally, a single dosage may wear off and or be washed away from the affected area so as to limit the relief provided to the affected area.

Other conventional methods of treating a sore throat has with throat lozenges.

Throat lozenges typically comprise a medicated sweet which may be dissolved in the mouth of the individual with the sore throat such that the medicine may come into contact with the affected area of the throat of the user when they swallow the dissolved lozenge. The lozenge is intended to remain in the mouth of the user once they begin us thereof so as to not contaminate the lozenge. Disadvantageously, such a lozenge in the mouth of the user may interfere with other activities of the user, such as eating, talking or the like. Additionally, once a lozenge has begun to be use, the amount of time for which it will last is limited and to at least a degree outside of the control of the user. Therefore, for the user to continue to sooth their sore throat, it will be necessary for the user to continue to apply further lozenges as necessary.

### SUMMARY OF THE INVENTION

According to a first embodiment of the present invention there is disclosed a composition for relieving a symptom of a sore throat of a user comprising an aqueous base and an effective amount of a throat soreness relieving agent.

The relieving agent may comprise menthol. The relieving agent may be included in a composition of between 0.001% w/v and 0.02% w/v. The relieving agent may be included in a composition of between 0.002% w/v and 0.004% w/v.

According to a further embodiment of the present invention there is disclosed a beverage comprising an aqueous base and including between 0.001% w/v and 0.02% w/v of a throat soreness relieving agent. The relieving agent may comprise menthol.

According to a further embodiment of the present invention there is disclosed a method for the treatment of cough in humans or animals in need of such relief comprising the administration of a safe and effective amount of the composition of claim 1.

Other aspects and features of the present invention will become apparent to those ordinarily skilled in the art upon review of the following description of specific embodiments of the invention in conjunction with the accompanying description.

### DETAILED DESCRIPTION

The present invention is composition to relieve of sore throats and the like utilizing a low concentration of a medicament in a beverage for consumption by a user. The composition includes the medicament in a sufficiently low enough concentration so as to be less detectable by a consumer and also able to be consumable in a higher volume than conventional cough syrup. Various terms relating to the methods and other aspects of the present invention are used throughout the present description. Such terms are to be given their ordinary meaning in the art unless otherwise indicated. Other specifically defined terms are to be construed in a manner consistent with the definition provided herein.

The amount of medicament incorporated in the composition may vary but generally will comprise from about 0.001% w/v to about 0.02% w/v of the total weight of the composition, with an amount of between 0.002 and 0.004% w/v being found to be particularly useful, or in other words an amount correspond to an equivalent amount of medicament from a conventional cough lozenge or dosage of cough syrup dissolved in liquid of between 50 to 500 ml.

The compositions of the present invention preferably include at least one topical anesthetics as an active component or medicament, such as, by way of non-limiting example phenol, lidocaine, dyclonine, benzocaine, menthol, benzyl alcohol, 30 salicyl alcohol, and hexylresorcinol, their pharmaceutically acceptable salts, and mixtures thereof. Optionally, the medicament may comprise other oral pharmacological active medicaments preferably selected from analgesic agents, decongestants, expectorants, antitussives, antihistamines and gastrointestinal agents.

In preparing the composition, the active component is incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. The most preferred carrier is a suspension of the pharmaceutical composition in an aqueous vehicle. Such suspending agents are well known to those skilled in the art. While the amount of water in the compositions of this invention can vary over quite a wide range depending upon the total weight and volume of the active component and other optional non-active ingredients, the total water content will generally comprise substantially the majority of the composition.

Although water itself may make up the entire carrier, typical liquid formulations preferably contain a co-solvent, for example, propylene glycol, corn syrup, glycerin, sorbitol solution and the like, to assist solubilization and incorporation of water-insoluble ingredients, such as flavoring agents and the like into the composition as may be deemed desirable by a consumer. The flavoring agents that may be used include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, oil of wintergreen (methyl salicylate), peppermint oil, clove oil, bay oil, eucalyptus oil, thyme oil, cedar leaf oil, oil of nutmeg, oil of sage, and oil of bitter almond. Also useful are artificial, natural or synthetic fruit flavors such as vanilla, and citrus oil, including lemon, orange, grape, lime and grapefruit and fruit essences including apple, pear, peach, strawberry, raspberry, cherry, plum, pineapple, apricot and so forth. These flavorings may be used individually or in admixture.

The present invention also provides a method of treating sore throats and other ailments such as by way of non-limiting example, laryngopharyngitis, by administering to a patient, an effective therapeutic amount of an active component contained in a low concentration within a beverage. As used from treatment of sore throats and the like.

“Effective amount” refers to an amount of a compound, material, or composition, as described herein that is effective to achieve a particular biological result. Such results include, but are not limited to, at least one of the following: reduced throat pain and irritation, reduced cough reduced phlegm and congestion. In various embodiments, “effective amount” refers to an amount suitable to reverse, reduce, prevent, or delay a decline in the above qualities.

In some embodiments of the present invention, the composition may be administered to the patient orally. As used herein, the term “oral administration” or “orally administering” means that the patient ingests, or a caretaker is directed to feed, or does feed, the patient one or more of the compositions described herein. In some embodiments, the composition may be orally provided to the patient, as a single or multiple serving beverage of any known volume, such as by way of non-limiting example, a standard bottle or can size. The patient may thereafter consume the composition as desired and at a pace as desired by the patient to relieve their symptoms associated with a throat infection or other throat irritation. It will be appreciated that the composition according to a the present description will permit the patient to consume or otherwise be provided with a dosage of cough or throat medicament or active component at a rate that corresponds more closely to the level of discomfort that is currently being experienced by them and will also enable the patient to provide to themselves a more even distribution of the medicament or active component to the affected area.

While specific embodiments of the invention have been described and illustrated, such embodiments should be considered illustrative of the invention only and not as limiting the invention as construed in accordance with the above description.

What is claimed is:

1. A composition for relieving a symptom of a sore throat of a user comprising an aqueous base and an effective amount of a throat soreness relieving agent.

2. The composition of claim 1 wherein said relieving agent comprises menthol.

3. The composition of claim 1 wherein said relieving agent is included in a composition of between 0.001% w/v and 0.02% w/v.

4. The composition of claim 1 wherein said relieving agent is included in a composition of between 0.002% w/v and 0.004% w/v.

5. A beverage comprising a water base and including between 0.001% w/v and 0.02% w/v of a throat soreness relieving agent.

6. The beverage of claim 5 wherein said relieving agent comprises menthol.

7. A method for the treatment of cough in humans or animals in need of such relief, comprising the administration of a safe and effective amount of the composition of claim 1.
